(19) United States
(12) Patent Application Publication
(10) Pub. No.: US 2025/0050109 A1
(43) Pub. Date: Feb. 13, 2025

(54) SYSTEMS AND METHODS FOR COORDINATING THERAPIES

(71) Applicant: Boston Scientific Neuromodulation Corporation, Valencia, CA (US)

(72) Inventors: Jianwen Gu, Richmond, TX (US); Rosana Esteller, Santa Clarita, CA (US)

(21) Appl. No.: 18/794,723

(22) Filed: Aug. 5, 2024

Related U.S. Application Data
(60) Provisional application No. 63/531,192, filed on Aug. 7, 2023.

(51) Int. Cl. A61N 1/36; A61N 1/02

(52) U.S. Cl. CPC A61N 1/36135; A61N 1/36053; A61N 1/36064; A61N 1/36082; A61N 1/36103; A61N 1/025

(57) ABSTRACT

A system may be configured for treating a condition and may include at least one therapy delivery system, at least one event detector and a controller. The therapy delivery system(s) may be configured to deliver at least two therapies to at least two therapy targets including deliver a first therapy to a first therapy target and deliver a second therapy to a second therapy target. The event detector(s) may be configured to detect at least one predefined event. The controller may be configured to coordinate the therapies based on the detected predefined event(s) using at least one defined event-therapy relationship.

### CLAIM OF PRIORITY

This application claims the benefit of U.S. Provisional Application No. 63/531,192, filed on Aug. 7, 2023, which is hereby incorporated by reference in its entirety.

### TECHNICAL FIELD

This document relates generally to medical systems, and more particularly, but not by way of limitation, to systems, devices, and methods for coordinating therapies based on detected predefined event(s) and a defined event-therapy relationship(s).

### BACKGROUND

Medical devices may include therapy-delivery devices configured to deliver a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc. By way of example and not limitation, a DBS system may be configured to treat tremor, bradykinesia, and dyskinesia and other motor disorders associated with Parkinson's Disease (PD). DBS therapy has been proposed to treat other conditions including dementia. An example of a PNS system is a vagal nerve stimulation (VNS) system. VNS may include stimulation of the cervical vagus and/or may include stimulation of a branch of the vagus nerve such as the auricular nerve. VNS has been proposed as an external stimulator (e.g., TENs) over the auricular nerve, and has been proposed as an implanted device (e.g., cervical vagal nerve implant).

Some conditions continue to be difficult to treat. It is therefore desirable to improve therapies to provide improved patient outcomes.

### SUMMARY

An example (e.g., Example 1) of a system may be configured for treating a condition and may include at least one therapy delivery system, at least one event detector and a controller. The therapy delivery system(s) may be configured to deliver at least two therapies to at least two therapy targets including deliver a first therapy to a first therapy target and deliver a second therapy to a second therapy target. The event detector(s) may be configured to detect at least one predefined event. The controller may be configured to coordinate the therapies based on the detected predefined event(s) using at least one defined event-therapy relationship.

In Example 2, the subject matter of Example 1 may optionally be configured such that the at least one therapy delivery system is configured to deliver a deep brain stimulation (DBS) therapy to a DBS target and to deliver one or both of a vagus nerve stimulation therapy (VNS) to a vagal nerve target or a spinal cord stimulation therapy (SCS) to a target in or near a spinal cord.

In Example 3, the subject matter of any one or more of Examples 1-2 may optionally be configured such that the at least one therapy delivery system is configured to deliver a deep brain stimulation (DBS) therapy to at least two different DBS targets

In Example 4, the subject matter of any one or more of Examples 1-3 may optionally be configured such that the at least one therapy delivery system is configured to deliver a vagus nerve stimulation therapy (VNS) to at least two different VNS targets.

In Example 5, the subject matter of any one or more of Examples 1-4 may optionally be configured such that the at least one therapy delivery system is configured to deliver an epilepsy therapy to treat an epileptic condition.

In Example 6, the subject matter of Example 5 may optionally be configured such that the at least one event detector is configured to receive user input and identify a predefined event related to the epileptic condition using the user input.

In Example 7, the subject matter of any one or more of Examples 5-6 may optionally be configured such that the at least one event detector is configured to sense electrical signals in a brain to detect a predefined event related to the epileptic condition.

In Example 8, the subject matter of any one or more of Examples 5-7 may optionally be configured such that the at least one event detector is configured to sense movement or lack of movement to detect a predefined event related to the epileptic condition.

In Example 9, the subject matter of any one or more of Examples 5-8 may optionally be configured such that the event detector is configured to analyze an image of a patient to detect the predefined event related the epileptic condition.

In Example 10, the subject matter of any one or more of Examples 1-9 may optionally be configured such that the at least one therapy delivery system is configured to deliver both VNS therapy and DBS therapy, and the controller is configured to coordinate the VNS therapy and the DBS therapy based on the detected at least one event.

In Example 11, the subject matter of Example 10 may optionally be configured such that the detected at least one event includes at least a first stage and a second stage for progression of the epileptic condition, and the at least one therapy delivery system is configured to coordinate the VNS therapy and the DBS therapy to provide a first therapy for the first stage and a second therapy for the second stage.

In Example 12, the subject matter of Example 10 may optionally be configured such that the at least one event detector is configured to detect a seizure event and to detect when the seizure event ended, and the at least one therapy delivery system is configured to deliver the first therapy during the seizure event and the second therapy after the seizure event.

In Example 13, the subject matter of any one or more of Examples 1-12 may optionally be configured such that the at least one therapy delivery system is configured to deliver a dementia therapy.

In Example 14, the subject matter of Example 13 may optionally be configured such that the at least one therapy delivery system is configured to deliver a deep brain stimulation (DBS) therapy to a DBS target and to deliver a vagus nerve stimulation therapy (VNS) to a vagal nerve target, the detected at least one event includes a cognitive task or a motor task, and the controller is configured to coordinate the VNS therapy and the DBS therapy based on the cognitive task or the motor task.

In Example 15, the subject matter of any one or more of Examples 1-14 may optionally be configured such that the at least one therapy delivery system is configured to deliver a stroke therapy.

Example 16 includes subject matter (such as a method, means for performing acts, machine readable medium including instructions that when performed by a machine cause the machine to perform acts, or an apparatus to perform). The subject matter may treat a condition and may include using at least one event detector to detect at least one predefined event, and using a controller configured to use at least one defined event-therapy relationship and the detected at least one predefined event to coordinate at least two therapies delivered to at least two therapy targets. The at least two therapies include a first therapy delivered to a first therapy target and a second therapy delivered to a second therapy target.

In Example 17, the subject matter of Example 16 may optionally be configured such that the at least two therapies include a deep brain stimulation (DBS) therapy to a DBS target, and further include one or both of a vagus nerve stimulation therapy (VNS) to a vagal nerve target or a spinal cord stimulation therapy (SCS) to a target in or near a spinal cord.

In Example 18, the subject matter of any one or more of Examples 16-17 may optionally be configured such that the at least two therapies include a first deep brain stimulation (DBS) therapy to a first DBS target and a second DBS therapy to a second DBS target.

In Example 19, the subject matter of any one of Examples 16-18 may optionally be configured such that the first therapy includes a first vagal nerve stimulation (VNS) therapy to a first VNS target and the second therapy includes a second VNS therapy to a second VNS target.

In Example 20, the subject matter of any one of Examples 16-19 may optionally be configured such that the at least two therapies include an epilepsy therapy for an epileptic condition.

In Example 21, the subject matter of any one of Examples 16-20 may optionally be configured such that the at least one event detector is used to detect the at least one predefined event by receiving a user input and identifying a predefined event related to the epileptic condition using the user input.

In Example 22, the subject matter of any one of Examples 16-21 may optionally be configured such that the at least one event detector is used to detect the at least one predefined event by sensing electrical signals in a brain.

In Example 23, the subject matter of any one of Examples 16-22 may optionally be configured such that the at least one event detector is used to detect the at least one predefined event by sensing movement or lack of movement.

In Example 24, the subject matter of any one of Examples 20-23 may optionally be configured such that the at least one event detector is used to detect the at least one predefined event by analyzing an image of a patient to detect the predefined event related the epileptic condition.

In Example 25, the subject matter of any one of Examples 16-24 may optionally be configured such that the at least two therapies include a deep brain stimulation (DBS) therapy to a DBS target and one or both of a vagus nerve stimulation therapy (VNS) to a vagal nerve target or a spinal cord stimulation therapy (SCS) to a target in or near a spinal cord, and the controller is used to coordinate the VNS therapy and the DBS therapy based on the detected at least one event.

In Example 26, the subject matter of Example 25 may optionally be configured such that the detected at least one event includes at least a first stage and a second stage for progression of the epileptic condition. The at least one therapy delivery system may be configured to coordinate the VNS therapy and the DBS therapy to provide a first therapy for the first stage and a second therapy for the second stage.

In Example 27, the subject matter of any one of Examples 25-26 may optionally be configured such that the detected at least one event includes a detected seizure event and a detected end to the seizure event, and the controller is used to deliver the first therapy during the seizure event and the second therapy after the seizure event.

In Example 28, the subject matter of any one of Examples 16-28 may optionally be configured such that the at least two therapies include a dementia therapy for a dementia condition.

In Example 29, the subject matter of Example 28 may optionally be configured such that the at least two therapies include a deep brain stimulation (DBS) therapy to a DBS target and a vagus nerve stimulation therapy (VNS) to a vagal nerve target. The detected at least one predefined event may include a cognitive or a motor task, and the controller may be used to coordinate the DBS therapy and the VNS therapy based on the cognitive task or the motor task.

In Example 30, the subject matter of any one of Examples 16-29 may optionally be configured such that the at least two therapies include a stroke therapy for a stroke condition.

Example 31 includes subject matter (such as a method, means for performing acts, machine readable medium including instructions that when performed by a machine cause the machine to perform acts, or an apparatus to perform). The subject matter may include delivering at least two therapies to at least two different therapy targets to provide therapy data for the at least two therapies. The therapy data may include therapy configuration data. The subject matter may include providing condition data indicative of an effect that the delivered at least two therapies has on a treated condition. The subject matter may further include detecting a plurality of events to compile event data, and analyzing the event data, the therapy data and the condition data to determine whether one or more of the at least two therapies are effective in treating the condition when delivered in response to the one or more of the detected plurality of events and to define one or more event-therapy relationships associating the one or more of the at least two therapies to be delivered in response to the one or more of the detected events.

In Example 32, the subject matter of Example 31 may optionally be configured such that the at least two therapies include a deep brain stimulation (DBS) therapy to a DBS target and a vagus nerve stimulation therapy (VNS) to a vagal nerve target, or include a first DBS therapy to a first DBS target and a second DBS therapy to a second DBS target.

In Example 33, the subject matter of any one of Examples 31-32 may optionally be configured such that each of the at least two therapies is delivered using different therapy parameters. Analyzing the event data, the therapy data and the condition data may include determining whether the different therapy parameters are effective in treating the condition in response to the one or more of the detected plurality of events.

In Example 34, the subject matter of any one of Examples 31-33 may optionally be configured to further include using machine learning to adjust therapy parameters for at least one of the at least two therapies based on the determined effectiveness until the adjusted therapy parameters are effective in treating the condition when delivered in response to the one or more of the detected plurality of events.

In Example 35, the subject matter of any one of Examples 31-34 may optionally be configured such that the detected plurality of events includes at least a first stage and a second stage for progression of an epileptic condition, and the defined one or more event-therapy relationships associate the one or more of the least two therapies to be delivered for at least the first stage and the second stage, or may optionally be configured such that the detected plurality of events includes a detected seizure event and a detected end to the seizure event, and the one or more event-therapy relationships, and the defined one or more event-therapy relationships associate the one or more of the least two therapies to be delivered for at least the seizure event and the end to the seizure event.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver neurostimulation such as DBS or SCS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a computing device for programming or controlling the operation of an electrical stimulation system.

FIG. 5 illustrates, by way of example and not limitation, a more generalized example of a medical system that includes a medical device and a processing system.

FIG. 6 illustrates, by way of example, an example of an electrical therapy-delivery system.

FIG. 7 illustrates, by way of example and not limitation, a monitoring system and/or the electrical therapy-delivery system of FIG. 6, implemented using an IMD.

FIG. 8 illustrates, by way of example and not limitation, a neuromodulation device with preset programs for delivering neuromodulation.

FIG. 9 illustrates, by way of example and not limitation, neural stimulation targets available for coordinated therapy.

FIG. 10 illustrates, by way of example and not limitation, a therapy device with a sensing function.

FIG. 11 illustrates, by way of example and not limitation, an external system with sensing function.

FIG. 12 illustrates, by way of example and not limitation, a system for treating a condition using multiple target therapy delivery systems and at least one event detector.

FIG. 13 illustrates, by way of example and not limitation, a system configured for defining event-therapy relationship(s) associating therapies to detected events.

FIG. 14 illustrates, by way of example and not limitation, a system for treating a condition associated with epilepsy using multiple target therapy delivery systems and at least one event detector to detect seizure-related events or other events associated with epilepsy.

FIG. 15 illustrates, by way of example and not limitation, a system configured for defining event-therapy relationship(s) associating epilepsy therapies to detected seizure-related events.

FIG. 16 illustrates, by way of example and not limitation, a system for treating a condition associated with dementia using multiple therapies and at least one event detector for detecting dementia-related events.

FIG. 17 illustrates, by way of example and not limitation, a system configured for defining event-therapy relationship(s) associating dementia therapies to detected dementia-related events.

FIG. 18 illustrates, by way of example and not limitation, a system for treating a condition associated with stroke using multiple therapies and at least one event detector for detecting stroke-related events.

FIG. 19 illustrates, by way of example and not limitation, a system configured for defining event-therapy relationship(s) associating stroke therapies to detected stroke-related events.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The present subject matter provides systems, devices and methods for using and/or developing coordinated therapies to treat a condition. Different therapies, alone or in combination with each other, may be implemented in response to different conditions. These different therapies may be delivered to different neural targets and may provide different mechanisms of action to treat a condition. Temporal parameters (e.g., frequency, pulse width, stimulation burst duration for a train of pulses, stimulation on/off timing and the like) and/or spatial parameters (e.g., stimulation amplitude, activated electrodes, polarity of active electrodes, and distribution of energy (fractionalization) across the active electrodes, and the like) for therapies may be adjusted for events detected using inputs (e.g., sensed parameters and/or user inputs) into the system. The system is capable of coordinating therapy delivery to address different detected events.

For example, two or more therapies may be applied to a patient who has epilepsy. The therapies may be selected and timed to ameliorate the patient condition (e.g., interrupt the progression of patient states that may develop into a full seizure). For example, a first therapy (e.g., VNS or DBS) may be provided when a first state of the epileptic patient is detected and a second therapy, another VNS and/or DBS) may be provided when a second state of the epileptic patient is detected. In another example, a therapy (e.g., DBS therapy, SCS or/VNS) may be delivered upon the detection of a seizure or known precursor to a seizure, and another therapy (DBS, SCS and/or VNS) may be delivered upon the termination of the seizure. For example, a first therapy may be delivered prophylactically to reduce the number or severity of seizures, a second therapy may be delivered to reduce the duration or intensity of a seizure that is currently occurring, and the third therapy may be delivered after the seizure has terminated (e.g., VNS to assist with relaxing the patient after the seizure) before returning the prophylactic therapy again.

Dementia is discussed herein as another example. Currently, there are not great treatments for dementia. Nucleus basalis of Meynert (NBM) stimulation and vagus nerve stimulation (VNS) have been shown to individually improve cognition. The NBM is part of the basal forebrain and is the major source of acetylcholine for the cortex. This cholinergic innervation appears to be important for cognition and learning. VNS, when paired with a successful motor task outcome, may enhance motor learning via cholinergic signaling in the basal forebrain. Combined NBM DBS stimulation and vagus nerve stimulation may provide greater benefit than either one alone, and combined NBM DBS stimulation and spinal cord stimulation may provide greater benefit than either one alone. DBS, implemented alone, may be paired with a task such that a particular DBS therapy is performed in response to the task. It is believed that pairing stimulation with a cognitive learning or memory task may further enhance cognition/memory. The tasks may be performed on a patient remote control, mobile phone, or computer. The user (e.g., patient or caregiver) may trigger stimulation through a remote control when they are going to do specific memory or cognitive task.

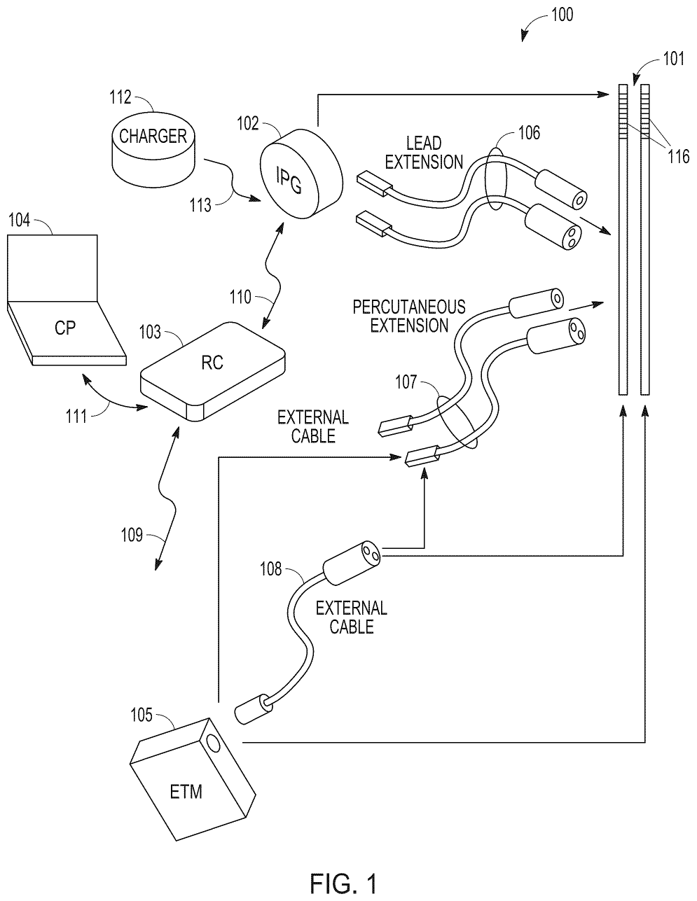

FIG. 1 illustrates, by way of example and not limitation, an electrical stimulation system 100, which may be used to deliver neurostimulation such as DBS or SCS. The electrical stimulation system 100 may generally include a one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. Other types of leads may be used. The IPG 102 includes pulse generation circuitry that delivers electrical modulation energy in the form of a pulsed electrical waveform (i.e., a temporal series of electrical pulses) to the electrodes in accordance with a set of modulation parameters.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneously charge the IPG 102 via a wireless link such as an inductive link 113. Once the IPG 102 has been programmed and its power source has been charged by the external charger or otherwise replenished, the IPG 102 may function as programmed without the RC 103 or CP 104 being present.

VNS systems are also configured to deliver neurostimulation using electrical waveforms. The VNS systems may include leads with electrode(s) configured to target the vagal targets. One example is a cuff electrode. Other examples include transvascular leads, subcutaneous leads implanted adjacent to the targeted nerve, or transcutaneous electrode(s) (TENS) positioned on the skin over the targeted nerve. However, other electrode configurations may be used to target the entire nerve or select axons in the nerve. For example, the cervical vagus nerve has a large number of fibers. These fibers have different diameters. Some fibers are myelinated and others are unmyelinated. These fibers may include A-Fibers (myelinated fibers with a diameter between 5-20 μm), B-fibers myelinated fibers with a diameter between 1-3 μm, and C-fibers (unmyelinated fibers with a diameter between 0.2-2 μm). Various embodiments may be configured to stimulate (e.g., generate action potentials) certain subsets of these fibers and/or inhibit or block actions potentials in some subsets of these fibers. The VNS system may be configured to target the cervical vagus nerve (left or right). For example, left cervical VNS has been used as a therapy for epilepsy. The vagus nerve includes many branches. The VNS system may be configured to target a vagus nerve branch. Examples of vagus nerve branches that may be targeted include but are not limited to the auricular nerve, the pharyngeal nerve, laryngeal nerves and superior and inferior cardiac nerves. The VNS system may be configured to be implantable or external.

VNS is an example of stimulation of an autonomic nerve. Various therapies may target other autonomic neural targets. The autonomic nervous system (ANS) regulates “involuntary” organs, while the contraction of voluntary (skeletal) muscles is controlled by somatic motor nerves. Examples of involuntary organs include respiratory and digestive organs, Often, the ANS functions in an involuntary, reflexive manner to regulate glands, to regulate muscles in the skin, eye, stomach, intestines and bladder, and to regulate cardiac muscle and the muscle around blood vessels, for example. The ANS includes, but is not limited to, the sympathetic nervous system and the parasympathetic nervous system. The sympathetic nervous system is affiliated with stress and the “fight or flight response” to emergencies. Among other effects, the “fight or flight response” increases blood pressure and heart rate to increase skeletal muscle blood flow and decreases digestion to provide the energy for “fighting or fleeing.” The parasympathetic nervous system is affiliated with relaxation and the rest and digest response” which, among other effects, decreases blood pressure and heart rate, and increases digestion to conserve energy. The ANS maintains normal internal function and works with the somatic nervous system.

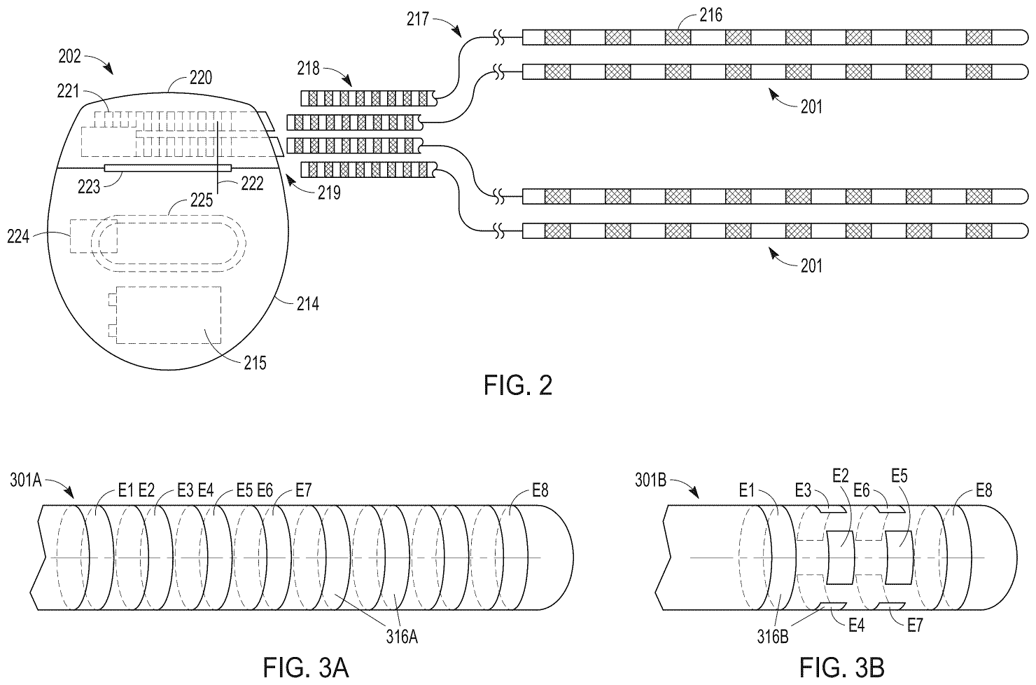

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 can also lack a battery and can be wirelessly powered by an external source. The IPG 202 may be coupled to one or more leads, such as leads 201 as illustrated herein. The leads 201 can each include a plurality of electrodes 216 for delivering electrostimulation energy, recording electrical signals, or both. In some examples, the leads 201 can be rotatable so that the electrodes 216 can be aligned with the target neurons after the neurons have been located such as based on the recorded signals. The electrodes 216 can include one or more ring electrodes, and/or one or more sets of segmented electrodes (or any other combination of electrodes), examples of which are discussed below with reference to FIGS. 3A and 3B.

The leads 201 can be implanted near or within the desired portion of the body to be stimulated. In an example of operations for DBS, access to the desired position in the brain can be accomplished by drilling a hole in the patient's skull or cranium with a cranial drill (commonly referred to as a burr), and coagulating and incising the dura mater, or brain covering. A lead can then be inserted into the cranium and brain tissue with the assistance of a stylet (not shown). The lead can be guided to the target location within the brain using, for example, a stereotactic frame and a microdrive motor system. In some examples, the microdrive motor system can be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header can comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 can include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 225 may also include a Radio-Frequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, the IPG 202 is typically implanted under the patient's clavicle (collarbone). The leads 201 (which may be extended by lead extensions, not shown) can be tunneled through and under the neck and the scalp, with the electrodes 216 implanted through holes drilled in the skull and positioned for desired target(s) for DBS therapy. The IPG 202 can also be implanted underneath the scalp closer to the location of the electrodes' implantation. The leads 201, or the extensions, can be integrated with and permanently connected to the IPG 202 in other solutions.

Stimulation in IPG 202 is typically provided by pulses each of which may include one phase or multiple phases. For example, a monopolar stimulation current can be delivered between a lead-based electrode (e.g., one of the electrodes 216) and a case electrode. A bipolar stimulation current can be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes can either be used (an active electrode) or unused (OFF). When the electrode is used, the electrode can be used as an anode or cathode and carry anodic or cathodic current. In some instances, an electrode might be an anode for a period of time and a cathode for a period of time. These and possibly other stimulation parameters taken together comprise a stimulation program that the stimulation circuitry 224 in the IPG 202 can execute to provide therapeutic stimulation to a patient.

In some examples, a measurement device coupled to the muscles or other tissue stimulated by the target neurons, or a unit responsive to the patient or clinician, can be coupled to the IPG 202 or microdrive motor system. The measurement device, user, or clinician can indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 can direct stimulus current to a selected angular range around the lead.

Segmented electrodes can typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering can be performed not only along a length of the lead but also around a circumference of the lead. This provides precise three-dimensional targeting and delivery of the current stimulus to neural target tissue, while potentially avoiding stimulation of other tissue. In some examples, segmented electrodes can be together with ring electrodes. A lead which includes at least one or more segmented electrodes may be referred to as a directional lead. In an example, all electrodes on a directional lead can be segmented electrodes. In another example, there can be different numbers of segmented electrodes at different longitudinal positions.

Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length the lead 219.

DBS systems may be configured to independently modulate more than one DBS target to provide more than one DBS therapy. According to various embodiments, the DBS system may be configured to coordinate these DBS therapies, such as may appropriate in response to different detected events.

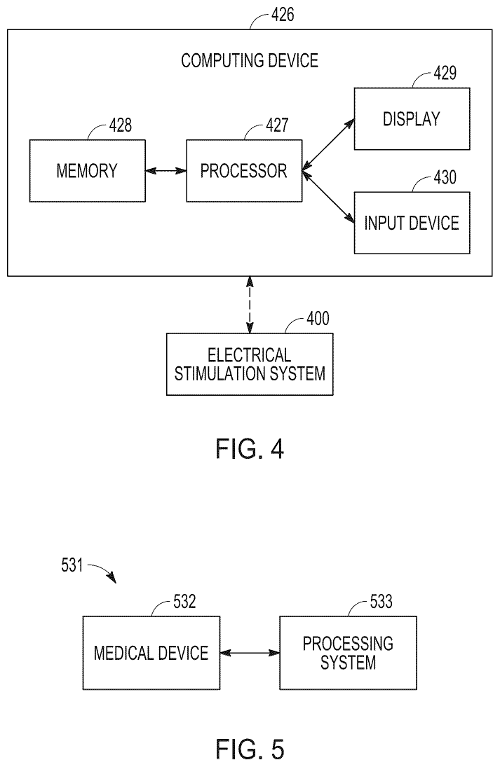

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1. Alternatively, the computing device 426 may be integrated with the electrical stimulation system 100, such as part of the IPG 102, RC 103, CP 104, or ETM 105 illustrated in FIG. 1.

The computing device 426, also referred to as a programming device, can be a computer, tablet, mobile device, or any other suitable device for processing information. The computing device 426 can be local to the user or can include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 can include a watch, wristband, smartphone, or the like. Such computing devices can wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, physical state or the like. In some examples, the computing device 426 may prompt the patient to take a periodic test (for example, every day) for cognitive ability to monitor, for example, progression of a disease that can impair cognitive function. In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 can be a wearable device used by the patient only during programming sessions. Alternatively, the computing device 426 can be worn all the time and continually or periodically adjust the stimulation parameters. In an example, a closed-loop algorithm for determining or updating stimulation parameters can be implemented in a mobile device, such as a smartphone, that is connected to the IPG or an evaluating device (e.g., a wristband or watch). These devices can also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a processor that can receive and execute a set of instructions of performing the functions, methods, or techniques described herein.

The memory 428 can store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information, and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and can include a printer. The display 429 may be a part of a user interface configured to display information about stimulation settings (e.g., electrode configurations and stimulation parameter values and value ranges) and user control elements for programming a stimulation setting into an IPG.

The input device 430 may be, for example, a keyboard, mouse, touch screen, track ball, joystick, voice recognition system, or any combination thereof, or the like. Another input device 430 may be a camera from which the clinician can observe the patient. Yet another input device 430 may include a microphone where the patient or clinician can provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user can provide information between the electrical stimulation system 400 and the computing device 426 using a computer-readable medium or by some other mechanism.

FIG. 5 illustrates, by way of example and not limitation, a more generalized example of a medical system 531 that includes a medical device 532 and a processing system 533. For example, the electrical stimulation system 400 of FIG. 4 may be a more specific example of the medical device 532 of FIG. 5, and computing device 426 of FIG. 4 may be a more specific example of the processing system 533 of FIG. 5. The medical device may be configured to provide sensing functions and/or therapy functions. For example, the medical device may include a device configured to use a parameter set to deliver an electrical stimulation therapy such as DBS, VNS and/or SCS. The medical device may be an implantable medical device such as an implantable neurostimulator. The implantable medical device may be configured to deliver SCS or DBS therapy. The medical device may include more than one medical device. The processing system may be within a single device or may be a distributed system across two or more devices including local and/or remote systems. According to various embodiments, the medical system may include at least one medical device configured to treat a condition by delivering a therapy to a patient.

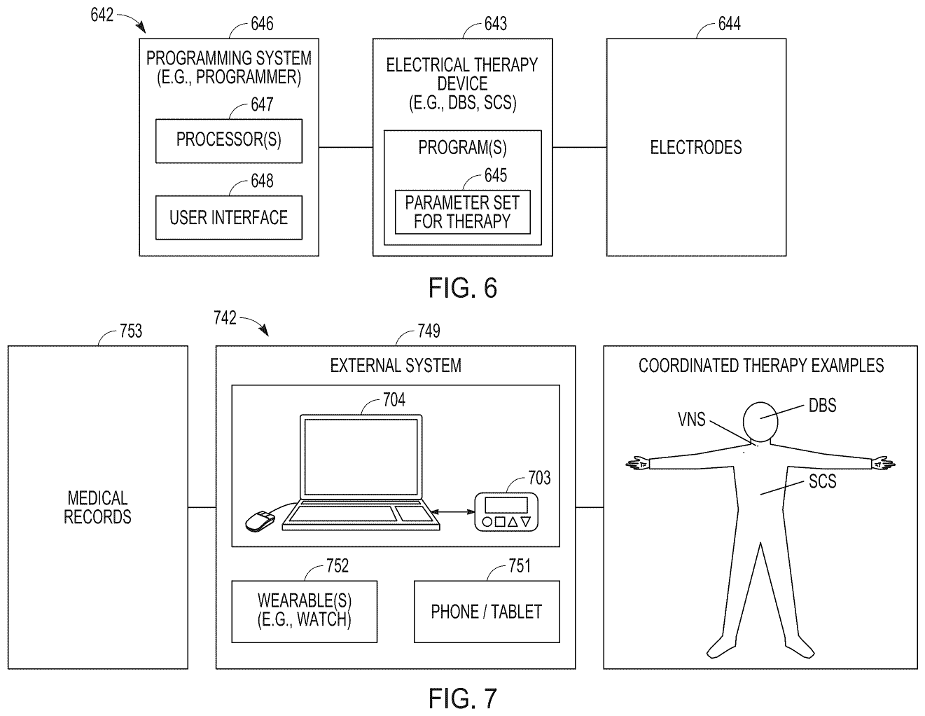

FIG. 6 illustrates, by way of example, an example of an electrical therapy-delivery system. The illustrated system 642 may be a more specific example of the system illustrated in FIG. 5, or form a portion of the system illustrated in FIG. 5. The illustrated system 642 includes an electrical therapy device 643 configured to deliver an electrical therapy to electrodes 644 to treat a condition in accordance with a programmed parameter set 645 for the therapy. The system 642 may include a programming system 646, which may function as at least a portion of a processing system, that may include one or more processors 647 and a user interface 648. The programming system 646 may be used to program and/or evaluate the parameter set(s) used to deliver the therapy. The illustrated system 642 may be a DBS system, a VNS system, a SCS system, or various combinations thereof.

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameters sets through a computerized programming system to allow the optimum modulation parameters to be determined based on patient feedback or other means and to subsequently program the desired modulation parameter sets.

FIG. 7 illustrates, by way of example and not limitation, the electrical therapy-delivery system of FIG. 6 implemented using one or more IMDs to provide VNS, DBS and/or SCS therapy (ies). These therapies may be coordinated to appropriately respond to the detection of different events. The illustrated system 742 includes an external system 749 that may include at least one programming device. The illustrated external system 749 may include a clinician programmer 704, similar to CP 104 in FIG. 1, configured for use by a clinician to communicate with and program the neuromodulator, and a remote control device 703, similar to RC 103 in FIG. 1, configured for use by the patient to communicate with and program the neuromodulator. For example, the remote control device 703 may allow the patient to turn a therapy on and off, change or select programs, and/or may allow the patient to adjust patient-programmable parameter(s) of the plurality of modulation parameters. The monitor and/or therapy device may be implemented using an implantable medical device and/or an external device such as a wearable device. The external system 749 may include a network of computers, including computer(s) remotely located from the IMD 750 that are capable of communicating via one or more communication networks with the programmer 704 and/or the remote control device 703. The remotely located computer(s) and the IMD 750 may be configured to communicate with each other via another external device such as the programmer 704 or the remote control device 703. The remote control device 703 and/or the programmer 704 may allow a user (e.g., patient, caregiver and/or clinician or rep) to answer questions as part of a data collection process. The external system 749 may include personal devices such as a phone or tablet 751, wearables such as a watch 752, sensors or therapy-applying devices. The watch may include sensor(s), such as sensor(s) for detecting activity, motion and/or posture. Other wearable sensor(s) may be configured for use to detect activity, motion and/or posture of the patient. The external system 749 may include, but is not limited to, a phone and/or a tablet. The system 742 may include medical record(s) 753 for the patient and broader patient population(s). The medical record(s) may be stored and accessed using one or more servers (e.g., local or remote servers such as cloud-based servers).

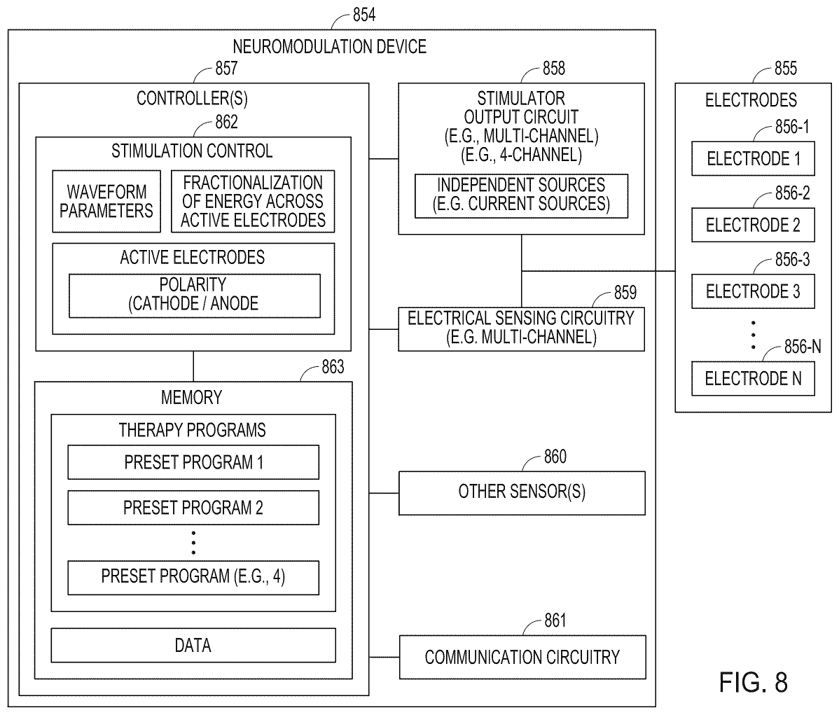

FIG. 8 illustrates, by way of example and not limitation, a neuromodulation device with preset programs for delivering neuromodulation. The neuromodulation device 854 may correspond to the implantable waveform generator 102 and/or the ETM 105 in FIG. 1, for example. The neuromodulation device 854 may be connected to a lead system 855 which includes one or more leads each configured to be electrically connected to the neuromodulation device 854 and a plurality of electrodes 856-1 to 856-N distributed in an electrode arrangement using the one or more leads. The illustrated neuromodulation device 854 includes one or more controllers 857 operably connected to a stimulator output circuit 858 to deliver neuromodulation to the electrodes. The stimulator output circuit 858 may include a plurality of independent sources such as independent current sources for each electrode. The stimulator output circuit 858 may be configured as a multi-channel (such as but not limited to four channels) system capable of simultaneously and independently generating and delivering separate stimulation waveforms to different electrode combinations. Some embodiments of the neuromodulation device 854 may include electrical sensing circuitry 859 configured to sense electrical activity (e.g., local field potentials, evoked compound actions potentials, evoked resonant neural activity (ERNA), electrospinogram, or other electrical signals) using at least some of the electrodes. Some embodiments of the neuromodulation device 854 may include other sensor(s) 860 that may be used to control the neuromodulation or provide context for the therapy or other events, or to detect events. Some embodiments of the neuromodulation device 854 may include communication circuitry 861 used to communicate with at least one external device. The controller(s) 857 may be configured to provide stimulation control 862 to control the neuromodulation generated by the stimulator output circuit 858 and delivered to the electrodes, which may include the waveform parameters for the neuromodulation, the active electrodes and polarity of the active electrodes used to deliver the neuromodulation, and the fractionalization of energy across the active electrodes. The controller(s) 857 may include memory 863 configured to store data and configured to store therapy programs. The therapy programs stored in the memory of the illustrated neuromodulation device 854 may include preset programs used to quickly determine that SCS lead(s) are placed in a position that enables the lead(s) to stimulate correct nerves for the therapy. For example, four programs may be stored. The four programs may correspond to four channels of the neuromodulator, which may be used to independently deliver stimulation to four different targets. The device 854 may be configured with more or fewer stimulation channels. Also, more than one program may be available for selection for each of the channel(s). Two or more different programs may switch the neurostimulation target, or two or more different programs may stimulate the same neurostimulation target using different stimulation parameters. The stored data in the memory 863 may include a variety of data used to deliver the therapy, to evaluate the delivered therapy, and/or to determine when events are detected and corresponding therapies to provide in response to the detection of the events. By way of example and not limitation, the data may include sensor data or operational data (e.g., impedance, battery charge, etc.) for the device.

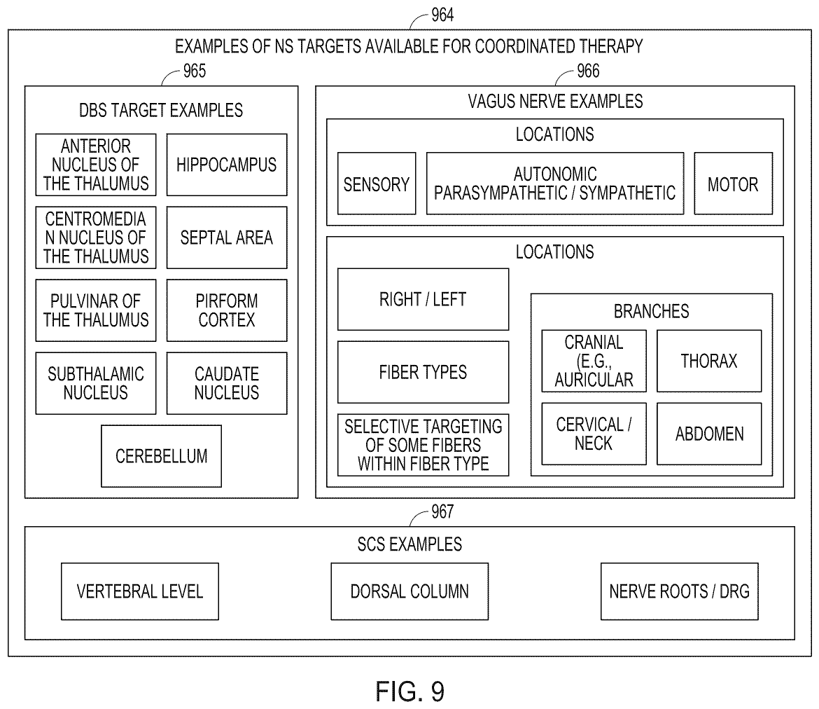

FIG. 9 illustrates, by way of example and not limitation, neural stimulation targets available for coordinated therapy. The system may be configured with one or more stimulation devices to stimulate multiple neural stimulation targets. Neural stimulation target examples 964 may include one or more DBS targets 965, one or more vagus nerve targets 966, and/or one or more SCS targets 967. Thus, for example, the system may be configured to deliver DBS therapies to at least two DBS targets or may be configured to deliver at least one DBS therapy to at least one DBS target and at least one VNS or SCS therapy to at least one VNS or SCS target. Examples of DBS targets 965 for deliver DBS therapy include the anterior nucleus of the thalamus, the centromedian nucleus of the thalamus, the pulvinar of the thalamus, the subthalamic nucleus, the hippocampus, the septal area, the piriform cortex, the caudate nucleus, the cerebellum and the nucleus basalis of Meynert (NBM). Examples of VNS targets 966 include sensory nerve fibers, motor fibers, or autonomic nerve fibers. Autonomic nerve fiber targets may include parasympathetic targets and/or sympathetic targets. Examples of VNS target location includes the right or left vagal nerve, the specific fiber types of the vagus nerve, the selective targeting of some fibers within a given fiber type (e.g., some but not all of the A-Fibers in the nerve, some but not all of the B-Fibers in the nerve, or some but not all of the C-Fibers in the nerve), and different branches of the vagus nerve. For example, therapy may be delivered to cranial branches (e.g., auricular), to branches in the cervical (neck) region, to branches in the thorax, or branches in the abdomen. Examples of vagus nerve branches that may be targeted include but are not limited to the auricular nerve, the pharyngeal nerve, laryngeal nerves and superior and inferior cardiac nerves. Examples of SCS target locations 967 include different vertebral levels, the dorsal column or portions of the dorsal column at different vertebral levels, and nerve roots or dorsal root ganglia at different vertebral levels.

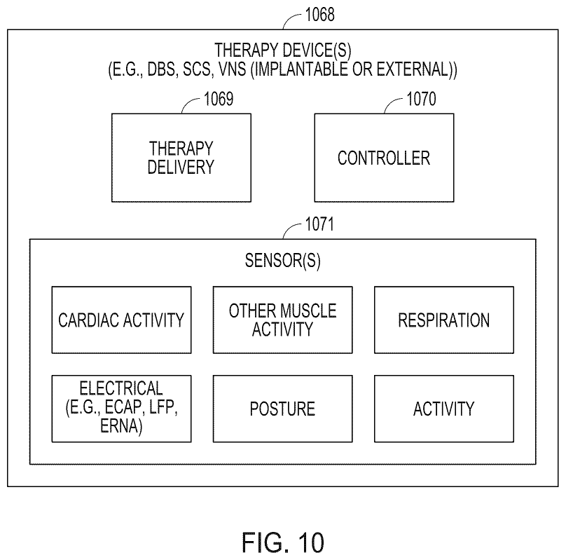

FIG. 10 illustrates, by way of example and not limitation, a therapy device with a sensing function. The therapy device may be configured to deliver DBS, SCS or VNS, and may be external or internal. The illustrated therapy device 1068 may include a therapy delivery circuit 1069 (e.g., waveform generator) and a controller 1070 configured to control the therapy delivery circuit to output the desired waveform. The therapy device 1068 may also include different sensors that may be used to provide feedback for therapy, to monitor a condition of the patient undergoing the therapy, or to trigger the start or stop of the therapy. By way of example and not limitation, the therapy device(s) may includes (include) sensors configured to detect cardiac activity such as but not limited to heart rhythm, heart rate, pulse and blood flow, muscle activity, respiration such as but not limited to respiration rate and volume, activity, posture or electrical activity such as ecaps, local field potentials or evoked resonant neural activity (ERNA). The sensor(s) of the therapy device(s) may be used to detect events to appropriately respond to the events.

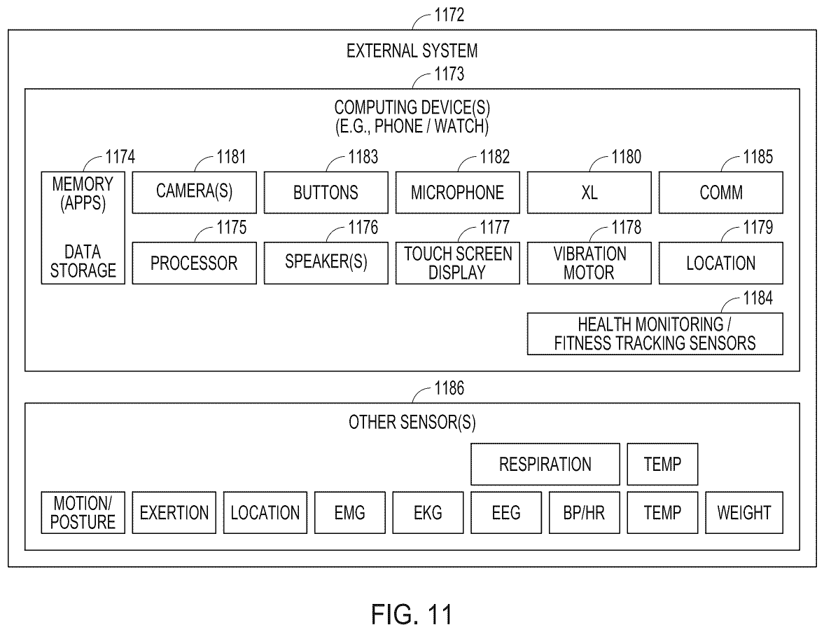

FIG. 11 illustrates, by way of example and not limitation, an external system with sensing function. The external system 1172 may include one or more computing devices 1173. The computing device(s) 1173 may include a phone, a tablet, and/or a wearable such as a watch, by way of example. The computing device(s) may include a number of features that may be used, alone or in combination with other features, by the system to detect events and/or respond to detected events. For example, the computing device(s) (1073 e.g., phone, watch, and the like) may include a processor 1174 and a memory 1175 including apps to be implemented by the processor to perform various processes. The memory 1175 may provide data storage for the external system. Other features of the computing device(s) 7073 may include at least one of speaker(s) 1176 to produce acoustic signals, a touch screen display 1177 for providing a user interface used to receive user inputs and provide visual outputs, and a vibration motor 1178 that may be used to provide a haptic output. The computing device(s) 1173 may include feature(s) capable of being used alone or in various combinations to detect an event 1079. For example, accelerometer(s) (XL) 1180, gyroscope, camera(s) 1181, microphone(s) 1182 and/or location service(s) 1183 may be used, with or without a clock and information about the patient's activities at different time(s) and/or location(s), to determine events. The location service(s) may use global positioning system (GPS), Wi-Fi and cellular towers (e.g., triangulation of Wi-Fi access points and/or cellular towers), Bluetooth beacons or Radio Frequency Identification (RFID). A determined location may be used to detect an event. For example, XLs 1180 can detect motion and/or posture. Microphones 1182 can be used to detect patient effort (breathing, grunts) and sound at the patient's location that may be used to detect an event. Cameras 1181 may be used to detect motion, location, and patient effort, such as via a shaky image, facial feature(s) of the patient, visual detection of an epileptic event, bradykinesia event or other event. The touch screen display 1177 may be used to determine attempted use of the computing device (e.g., phone) via an unlocked screen or other interaction with the device via the touch screen display or other button(s) 1183 on the device, which may be used to detect an event. The computing device(s) 1173 may include health monitoring/fitness tracking sensor(s) 1184 that may make use of other sensor(s) in the device. Health monitoring/fitness tracking sensor(s) 1184 may be configured to detect at least an estimate of heart rate, ECG, blood pressure, oxygen, steps, sleep, exercise, stress, and the like. A more exhaustive list of sensor(s) of computing device(s) may include any one or various combinations of an accelerometer, a gyroscope, a magnetometer/compass, a barometric pressure sensor, a body temperature sensor, a heart rate monitor, an oximetry sensor, an ambient light sensor, a bioimpedance sensor, a proximity sensor, an orientation sensor, a pedometer, a calorie counter, an ECG sensor, a gesture sensor, a UV sensor, an electrodermal activity sensor, a skin conductance sensor, and a GPS sensor.

The computing device(s) 1173 in the external system 1172 also includes a number of features that may be used to detect events and/or respond to a detected event. For example, the computing device(s) 1173 may include communication technology 1185 (e.g., Wi-Fi, Bluetooth) for use to communicate with other computing device(s), other sensor(s), and/or other perceptible signal transducer(s). Other sensor(s) 1186 may include other motion, exertion and/or posture sensors, other exertion sensor(s), other sensor(s) for detecting location (e.g., beacon, such as within range of a Bluetooth device), other sensors of physiological parameters such as EMG, EKG, EEG, respiration, galvanic skin response (GSR), cardiovascular parameters such blood pressure, rhythm and/or heart rate, temperature, and weight. The external system may include other perceptible signal transducer(s) such as audio device (e.g., speakers, headsets, earbuds, hearing aids), haptic device(s) (e.g., vibration motor or other devices for provide a tactile and/or kinesthetic sensation), and/or visual device(s) (e.g., lights, lasers, computer or television monitor, projection system, augmented reality or virtual reality).

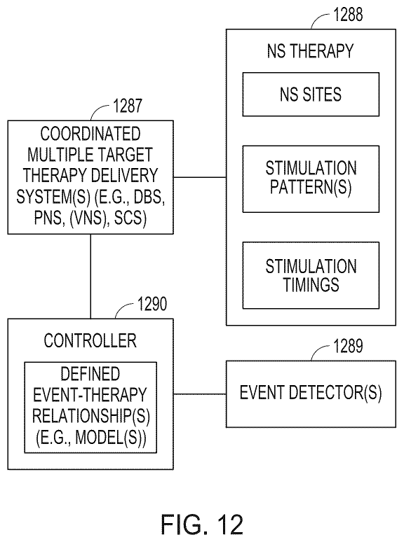

FIG. 12 illustrates, by way of example and not limitation, a system for treating a condition using multiple target therapy delivery systems and at least one event detector. The system may include at least one therapy delivery system 1287 configured to deliver at least two therapies 1288 to at least two therapy targets including deliver a first therapy to a first therapy target and deliver a second therapy to a second therapy target. The different therapies may include different therapy targets, different stimulation parameters (e.g., different pulse-to-pulse intervals, amplitudes, waveforms, as well as different number of pulses in different bursts of pulses, and different burst-to burst intervals, etc.), and different stimulation timing (start and stop times, as well as pulse frequency, pulse width, bursts of pulses, duty cycles between bursts, and relative timing between therapies). The therapy targets may include targets identified in FIG. 9 or may include other therapy targets. The same therapy system may be configured to deliver to the two targets (such as a DBS system configured to stimulate two or more DBS targets, a VNS system configured to stimulate two or more VNS targets, or a SCS system configured to stimulate or more SCS targets. Systems may be developed to provide one device configured to deliver stimulation to DBS target(s) and VNS target(s), to DBS target(s) and SCS target(s), to VSN target(s) and SCS target(s), or to DBS target(s), VNS target(s) and SCS target(s). Systems may be developed to provide direct communication (or indirect communication via another device such as a programmer, remote control, tablet or phone) between different devices to coordinate the therapies provided by the devices (e.g., communication links to a DBS device, to a VNS device, and/or to a SCS device. The system therapy delivery system 1287 is configured to coordinate the different therapies. The system may include at least one event detector 1289 configured to detect at least one predefined event. The event(s) may be determined to be relevant to the condition being treated by the therapy delivery system or otherwise relevant to the efficacy of the delivered therapy. The system may include a controller 1290 configured to coordinate the at least two therapies based on the detected at least one predefined event using at least one defined event-therapy relationship. For example, various models may be developed and used to determine the appropriate therapy (ies) that should be delivered in response to the event to treat condition(s) of the patient. The controller may be implemented in one or more of the therapy-delivery device(s) in the therapy-delivery system(s) 1287 or may be one or more separate controllers (e.g., programmer(s), remote control(s), phone(s), tablet(s) and the like) configured to communicate and with the therapy-delivery device(s) in the therapy delivery system 1287.

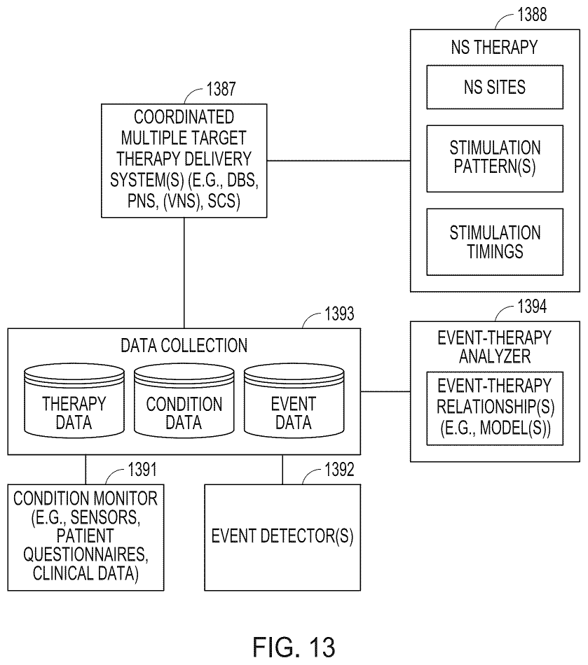

FIG. 13 illustrates, by way of example and not limitation, a system configured for defining event-therapy relationship(s) associating therapies to detected events. Similar to FIG. 12, the system includes at least one therapy delivery system 1387 configured to deliver at least two therapies 1388 to at least two therapy targets including deliver a first therapy to a first therapy target and deliver a second therapy to a second therapy target. The different therapies may include different therapy targets, different stimulation parameters (e.g., different pulse-to-pulse intervals, amplitudes, waveforms, as well as different number of pulses in different bursts of pulses, and different burst-to burst intervals, etc.), and different stimulation timing (start and stop times, as well as pulse frequency, pulse width, bursts of pulses, duty cycles between bursts, and relative timing between therapies), The therapy targets may include targets identified in FIG. 9, or may include other therapy targets. The same therapy system may be configured to deliver to the two targets (such as a DBS system configured to stimulate two or more DBS targets, a VNS system configured to stimulate two or more VNS targets, or a SCS system configured to stimulate or more SCS targets. Systems may be developed to provide one device configured to deliver stimulation to DBS target(s) and VNS target(s), to DBS target(s) and SCS target(s), to VSN target(s) and SCS target(s), or to DBS target(s), VNS target(s) and SCS target(s). Systems may be developed to provide direct communication (or indirect communication via a programmer, remote control, tablet or phone) between different devices to coordinate the therapies provided by the devices (e.g., communication links to a DBS device, to a VNS device, and/or to a SCS device. The system therapy delivery system 1387 is configured to coordinate the different therapies.

The system may include a condition monitor(s) 1391 to detect a patient condition that is the condition being treated by the therapies or a condition associated with the patient condition being treated or to the therapies being delivered to the patient (including side effects, comorbidities, medication/medication schedule, and the like). The system may also include at least one event detector(s) 1392 to detect predefined events. Machine learning (or other artificial intelligence) may be implemented to identify event(s) that appear to have an effect the patient or the efficacy of the therapy (ies) delivered to the patient. The system may include a data collection system 1393 configured to detect therapy data (e.g., therapy configuration data such as stimulation parameters, neural stimulation sites, stimulation patterns, stimulation timing, and the like) for the at least two therapy (ies), condition data from the condition monitor, and event data from the event detector(s). The event-therapy analyzer 1394 may be configured to use machine learning (or other artificial intelligence) to analyze the collected data, event data and condition data to identify event-therapy relationship(s) (e.g., develop models) that may be used by the controller 1290 in the system of FIG. 12 to coordinate the therapies in response to detected event(s). A plurality of events may be detected to compile event data. The event data, the therapy data, and the condition data may be analyzed to determine whether one or more of the at least two therapies are effective in treating the condition when delivered in response to the one or more of the detected plurality of events, and define one or more event-therapy relationships associating the one or more of the at least two therapies to be delivered in response to the one or more of the detected events. The event-therapy analyzer 1394 may operate to update a programming model by training, re-training, or updating a model (e.g., an artificial intelligence model, such as a neural network) based on the analyzed data. One or multiple instances of a model may be trained to generate programs and program parameters, for any one or more of two or more therapies. The models may be patient-specific or may be developed for a larger population.

As identified above, machine learning may be used to identify event-therapy relationships and/or may be used to identify the events that have an effect on the patient's condition or on the therapy being delivered to the patient. Machine-learning programs (MLPs), also referred to as machine-learning algorithms or tools, are utilized to perform operations associated with machine learning tasks, such as identifying relationship(s) in the collected data, including feature(s) in a sensed signal, different neurostimulation therapies, and waveform parameter(s) used to control the different neurostimulation therapies. Machine learning is a field of study that gives computers the ability to learn without being explicitly programmed. Machine learning explores the study and construction of algorithms that may learn from existing data (e.g., “training data”) and make predictions about new data. Such machine-learning tools may build a model from example training data in order to make data-driven predictions or decisions expressed as outputs or assessments. The machine-learning algorithms use the training data to find correlations among identified features that affect the outcome. The machine-learning algorithms use features for analyzing the data to generate assessments. A feature is an individual measurable property of the observed phenomenon. In the context of a biological signal, some examples of features may include, but are not limited to, peak(s) such as a minimum peak, a maximum peak as well as local minimum and maximum peaks, a range between peaks, a difference in values for features, a feature change with respect to a baseline, an area under a curve, a curve length, an oscillation frequency, and a rate of decay for peak amplitude. Inflection points in the signal may also be an observable feature of the signal, as an inflection point is a point where the signal changes concavity (e.g., from concave up to concave down, or vice versa), and may be identified by determining where the second derivative of the signal is zero. Detected feature(s) may be partially defined by time (e.g., length of curve over a time duration, area under a curve over a time duration, maximum or minimum peak within a time duration, etc.). The machine-learning algorithms use the training data to find correlations among the identified features that affect the outcome or assessment. With the training data and the identified features, the machine-learning tool is trained. The machine-learning tool appraises the value of the features as they correlate to the training data. The result of the training is the trained machine-learning program. Various machine learning techniques may be used to train models to make predictions based on data fed into the models. During a learning phase, the models are developed against a training dataset of inputs to optimize the models to correctly predict the output for a given input. A training data set may be defined for desired functionality of the closed-loop algorithm and closed loop parameters may be defined for desired functionality of the closed-loop algorithm. Generally, the learning phase may be supervised, semi-supervised, or unsupervised; indicating a decreasing level to which the “correct” outputs are provided in correspondence to the training inputs. In a supervised learning phase, all of the outputs are provided to the model and the model is directed to develop a general rule or algorithm that maps the input to the output. In contrast, in an unsupervised learning phase, the desired output is not provided for the inputs so that the model may develop its own rules to discover relationships within the training dataset. In a semi-supervised learning phase, an incompletely labeled training set is provided, with some of the outputs known and some unknown for the training dataset. Models may be run against a training dataset for several epochs (e.g., iterations), in which the training dataset is repeatedly fed into the model to refine its results. For example, in a supervised learning phase, a model is developed to predict the output for a given set of inputs and is evaluated over several epochs to more reliably provide the output that is specified as corresponding to the given input for the greatest number of inputs for the training dataset. In another example, for an unsupervised learning phase, a model is developed to cluster the dataset into groups and is evaluated over several epochs as to how consistently it places a given input into a given group and how reliably it produces the n desired clusters across each epoch.

Once an epoch is run, the models are evaluated, and the values of their variables are adjusted to attempt to better refine the model in an iterative fashion. In various aspects, the evaluations are biased against false negatives, biased against false positives, or evenly biased with respect to the overall accuracy of the model. The values may be adjusted in several ways depending on the machine learning technique used. For example, in a genetic or evolutionary algorithm, the values for the models that are most successful in predicting the desired outputs are used to develop values for models to use during the subsequent epoch, which may include random variation/mutation to provide additional data points. One of ordinary skill in the art will be familiar with several machine learning algorithms that may be applied with the present disclosure, including linear regression, random forests, decision tree learning, neural networks, deep neural networks, and the like. New data is provided as an input to the trained machine-learning program, and the trained machine-learning program generates the assessment as output. The assessment that is output may be out of an expected range (e.g., anomalous), indicating that remedial action such as retraining of the machine learning algorithm(s) is warranted. The system also may be configured to determine that the new data includes anomalous data with respect to the training data that was used to train the machine-learning program. The detection of new data that is anomalous may trigger remedial action(s) such as, if it is determined that the previously used training data is outdated, retraining the machine learning program using updated training data.

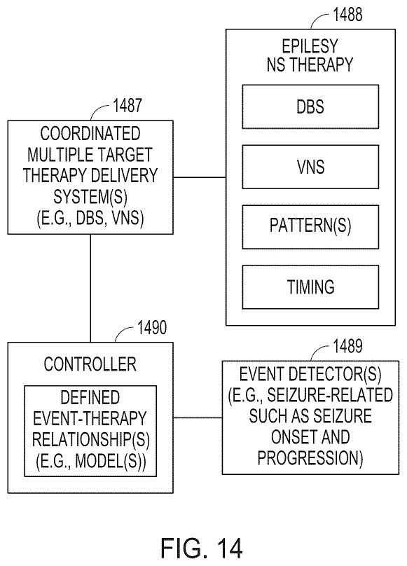

FIG. 14 illustrates, by way of example and not limitation, a system for treating a condition associated with epilepsy using multiple target therapy delivery systems and at least one event detector to detect seizure-related events or other events associated with epilepsy. The system may include at least one therapy delivery system 1487 configured to deliver at least two epilepsy-related therapies 1488 to at least two therapy targets including deliver a first therapy to a first therapy target and deliver a second therapy to a second therapy target. The different therapies may include different therapy targets, different stimulation parameters (e.g., different pulse-to-pulse intervals, amplitudes, waveforms, as well as different number of pulses in different bursts of pulses, and different burst-to burst intervals, etc.), and different stimulation timing (start and stop times, as well as pulse frequency, pulse width, bursts of pulses, duty cycles between bursts, and relative timing between therapies), The therapy targets may include targets identified in FIG. 9, or may include other therapy targets. For example, two or more therapies may be applied to a patient who has epilepsy. By way of example and not limitation, DBS targets of interest for epilepsy include the anterior nucleus of the thalamus, the centromedian nucleus of the thalamus, the hippocampus, the pulvinar of the thalamus, the piriform cortex, the septal area, the subthalamic nucleus, the cerebellum and the caudate nucleus. The therapies may be selected and timed to ameliorate the patient condition. For example, therapies may be selected in response to specific patient states to interrupt the progression of patient states that may develop into a full seizure. Preferably, a cascade of events leading to a seizure may be interrupted early with very little or no side effects. The cascade of events may be part of the model, and therapy may be delivered to a target in anticipation of and desire to prevent the next event in the cascade of events. For example, a first therapy (e.g., VNS or DBS) may be provided when a first state of the epileptic patient is detected and a second therapy, another VNS and/or DBS) may be provided when a second state of the epileptic patient is detected. In another example, a therapy (e.g., DBS therapy, SCS or/VNS) may be delivered upon the detection of a seizure or known precursor to a seizure, and another therapy (DBS, SCS and/or VNS) may be delivered upon the termination of the seizure. For example, a first therapy may be delivered prophylactically to reduce the number or severity of seizures, a second therapy may be delivered to reduce the duration or intensity of a seizure that is currently occurring, and the third therapy may be delivered after the seizure has terminated (e.g., VNS to increase the “rest and digest” autonomic response to assist with recovering from a seizure) before returning the prophylactic therapy again.

The system may include at least one event detector 1489 configured to detect at least one predefined event. The event(s) are relevant or determined to be potentially relevant to the epileptic patient being treated by the therapy delivery system. The system may include a controller 1490 configured to coordinate the at least two therapies based on the detected at least one predefined event using at least one defined event-therapy relationship. For example, various models may be developed and used to determine the appropriate therapy (ies) that should be delivered in response to the event to treat condition(s) of the patient. The controller may be implemented in one or more of the therapy-delivery device(s) in the therapy-delivery system(s) 1487 or may be one or more separate controllers (e.g., programmer(s), remote control(s), phone(s), tablet(s) and the like) configured to communicate and with the therapy-delivery device(s) in the therapy delivery system 1487.

Models may be used to describe seizure evolution (Liou J Y, Smith E H, Bateman L M, Bruce S L, McKhann G M, Goodman R R, Emerson R G, Schevon C A, Abbott L F. A model for focal seizure onset, propagation, evolution, and progression. Elife. 2020 Mar. 23; 9: e50927. doi: 10.7554/eLife.50927. PMID: 32202494; PMCID: PMC7089769., Karoly P J, Kuhlmann L, Soudry D, Grayden D B, Cook M J, Freestone D R. Seizure pathways: A model-based investigation. PLOS Comput Biol. 2018 Oct. 11; 14(10):e1006403. doi: 10.1371/journal.pcbi.1006403. PMID: 30307937; PMCID: PMC6199000.). In some embodiments, the models may be made patient specific. In some embodiments, the models are not specific to the patients but are more global to a larger patient population. Machine learning may be used to develop and optimize the models to terminate the seizure, depending on seizure type. For example, Liou et al. 2020 shows that spiral-wave activity, representing status epilepticus, which is a severe and dangerous condition, can be terminated by a global, synchronized excitatory input. The present subject matter may provide a similar input using multiple modes of stimulation in response to this detected condition. Seizure onset can predict its type/evolution (Donos C, Maliia M D, Dümpelmann M, Schulze-Bonhage A. Seizure onset predicts its type. Epilepsia. 2018 March: 59(3): 650-660. doi: 10.1111/epi. 13997. Epub 2018 Jan. 11. PMID: 29322500). A model may be developed to quickly provide the appropriate type of stimulation at seizure onset. The event detector(s) may be configured to use various inputs to determine seizure-related events such as seizure onset and progression. Video may be used, with or without other signals like brain activity, cardiac activity, accelerometry, to identify patient-specific motion signatures that indicate seizure onset and/or type of seizure to trigger stimulation (Ahmedt-Aristizabal D, Sarfraz M S, Denman S, Nguyen K, Fookes C, Dionisio S. Stiefelhagen R. Motion Signatures for the Analysis of Seizure Evolution in Epilepsy. Annu Int Conf IEEE Eng Med Biol Soc. 2019 July; 2019:2099-2105. doi: 10.1109/EMBC.2019.8857743. PMID: 31946315). Brain activity may be recorded during sleep-wake cycle to identify when seizures are most likely to happen such that appropriate stimulation may be delivered during these times of high seizure probability. Stimulation may be used to help normalize brain activity during the sleep-wake cycle to prevent seizures. (Bazil C W. Seizure modulation by sleep and sleep state. Brain Res. 2019 Jan. 15; 1703:13-17. doi: 10.1016/j.brainres.2018.05.003. Epub 2018 May 18. PMID: 29782849).

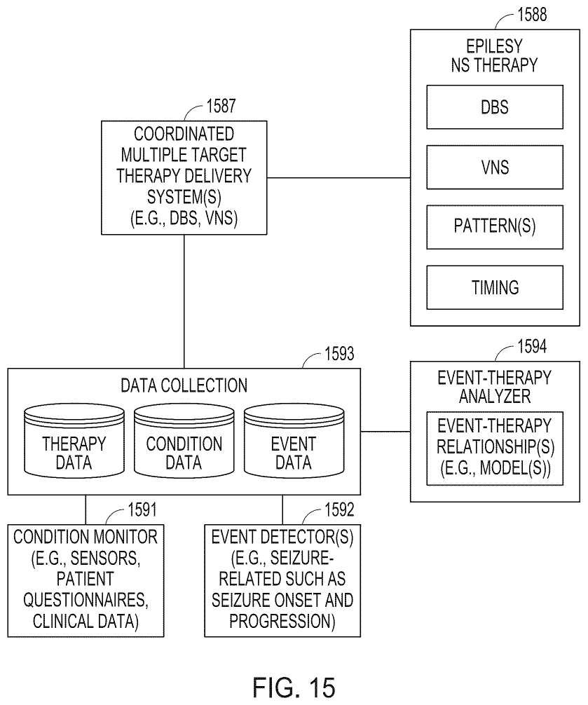

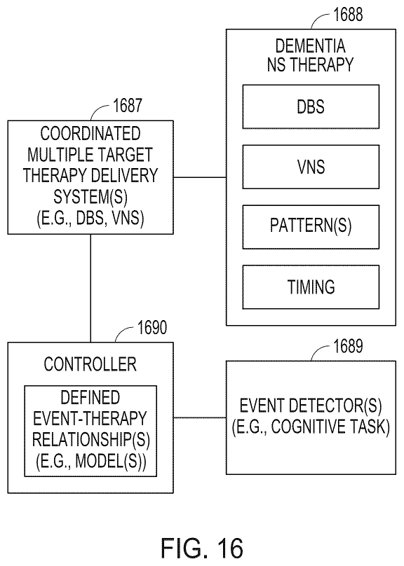

FIG. 15 illustrates, by way of example and not limitation, a system configured for defining event-therapy relationship(s) associating epilepsy therapies to detected seizure-related events. Similar to FIG. 14, the system includes at least one therapy delivery system 1587 configured to deliver at least two therapies 1588 to at least two therapy targets including deliver a first therapy to a first therapy target and deliver a second therapy to a second therapy target. The different therapies may include different therapy targets, different stimulation parameters (e.g., different pulse-to-pulse intervals, amplitudes, waveforms, as well as different number of pulses in different bursts of pulses, and different burst-to burst intervals, etc.), and different stimulation timing (start and stop times, as well as pulse frequency, pulse width, bursts of pulses, duty cycles between bursts, and relative timing between therapies), The therapy targets may include targets identified in FIG. 9, or may include other therapy targets. The same therapy system may be configured to deliver to the two targets (such as a DBS system configured to stimulate two or more DBS targets, a VNS system configured to stimulate two or more VNS targets, or a SCS system configured to stimulate or more SCS targets. Systems may be developed to provide one device configured to deliver stimulation to DBS target(s) and VNS target(s), to DBS target(s) and SCS target(s), to VSN target(s) and SCS target(s), or to DBS target(s), VNS target(s) and SCS target(s). Systems may be developed to provide direct communication (or indirect communication via a programmer, remote control, tablet or phone) between different devices to coordinate the therapies provided by the devices (e.g., communication links to a DBS device, to a VNS device, and/or to a SCS device. The system therapy delivery system 1587 is configured to coordinate the different epilepsy-related therapies.

The system may include a condition monitor(s) 1591 to detect an epileptic condition other conditions associated with the epileptic condition or to the therapies delivered to the patient (including side effects, comorbidities, medication/medication schedule, and the like). The system may also include at least one event detector(s) 1592 to detect predefined events. Machine learning may be implemented to identify event(s) that appear to have an effect the patient or the efficacy of the therapy (ies) delivered to the patient with epilepsy. The system may include a data collection system 193 configured to detect epilepsy therapy data (e.g., therapy configuration data such as stimulation parameters, neural stimulation sites, stimulation patterns, stimulation timing, and the like) for the at least two therapy (ies), condition data from the condition monitor, and event data from the event detector(s). The event-therapy analyzer 1594 may be configured to use machine learning to analyze the collected data, event data and condition data to identify event-therapy relationship(s) (e.g., models) that may be used by the controller 1490 in the system of FIG. 14 to coordinate the epilepsy therapies in response to detected event(s). A plurality of events may be detected to compile event data. The event data, the therapy data, and the condition data may be analyzed to determine whether one or more of the at least two epilepsy therapies are effective in treating the condition when delivered in response to the one or more of the detected plurality of events, and define one or more event-therapy relationships associating the one or more of the at least two therapies to be delivered in response to the one or more of the detected events.

FIG. 16 illustrates, by way of example and not limitation, a system for treating a condition associated with dementia using multiple therapies and at least one event detector for detecting dementia-related events. The system may include at least one therapy delivery system 1687 configured to deliver at least two therapies 1688 to at least two therapy targets including deliver a first therapy to a first therapy target and deliver a second therapy to a second therapy target. The different dementia-related therapies may include different therapy targets, different stimulation parameters (e.g., different pulse-to-pulse intervals, amplitudes, waveforms, as well as different number of pulses in different bursts of pulses, and different burst-to burst intervals, etc.), and different stimulation timing (start and stop times, as well as pulse frequency, pulse width, bursts of pulses, duty cycles between bursts, and relative timing between therapies), The therapy targets may include targets identified in FIG. 9, or may include other therapy targets. For example, two or more therapies may be applied to a patient who has dementia. The system may include at least one event detector 1689 configured to detect at least one predefined event. The event(s) are relevant or determined to be potentially relevant to the dementia being treated by the therapy delivery system. The system may include a controller 1690 configured to coordinate the at least two therapies based on the detected at least one predefined event using at least one defined event-therapy relationship. For example, various models may be developed and used to determine the appropriate therapy (ies) that should be delivered in response to the event to treat condition(s) of the patient. The controller may be implemented in one or more of the therapy-delivery device(s) in the therapy-delivery system(s) 1687 or may be one or more separate controllers (e.g., programmer(s), remote control(s), phone(s), tablet(s) and the like) configured to communicate and with the therapy-delivery device(s) in the therapy delivery system 1687.

Currently, there are not great treatments for dementia. Nucleus basalis of Meynert (NBM) stimulation and vagus nerve stimulation (VNS) have been shown to individually improve cognition. The NBM is part of the basal forebrain and is the major source of acetylcholine for the cortex. This cholinergic innervation appears to be important for cognition and learning. A recent paper (Bowles S, Hickman J, Peng X. Williamson W R. Huang R, Washington K, Doncgan D, Welle C G. Vagus nerve stimulation drives selective circuit modulation through cholinergic reinforcement. Neuron. 2022 Sep. 7; 110(17): 2867-2885.e7. doi: 10.1016/j.neuron.2022.06.017. Epub 2022 Jul. 19. PMID: 35858623; PMCID: PMC10212211) shows that VNS, when paired with a successful motor task outcome, may enhance motor learning via cholinergic signaling in the basal forebrain.

Combined NBM DBS stimulation and vagus nerve stimulation may provide greater benefit than either one alone. VNS may be invasive (e.g., implanted to stimulate the cervical vagus nerve) or non invasive (e.g., transcutaneous stimulation of the vagus or branch thereof such as the auricular nerve branch. Branches of the vagus nerve extend to specific locations such as nucleus of the solitary tract, locus coeruleus, and the basal forebrain. Combined NBM DBS stimulation and spinal cord stimulation may provide greater benefit than either one alone. DBS, implemented alone, may be paired with a task such that a particular DBS therapy is performed in response to the task. It is believed that pairing stimulation with a cognitive learning or memory task may further enhance cognition/memory. The tasks may be performed on a patient remote control, mobile phone, or computer. The user (e.g., patient or caregiver) may trigger stimulation through a remote control when they are going to do specific memory or cognitive task. Additionally or alternatively, brain activity sensors may be used to determine appropriate times to stimulate (Kahana M J, Ezzyat Y, Wanda P A, Solomon E A, Adamovich-Zeitlin R, Lega B C, Jobst B C, Gross R E, Ding K, Diaz-Arrastia R R. Biomarker-guided neuromodulation aids memory in traumatic brain injury. Brain Stimul. 2023 Jul. 5; 16(4): 1086-1093. doi: 10.1016/j.brs.2023.07.002. Epub ahead of print. PMID: 37414370.)

Machine learning algorithms may be used to select stimulation parameters/patterns/timing based on task performance and/or brain activity. Stimulation patterns may include intermittent tonic, intermittent sequences, burst (e.g., theta burst), and the like. Other potential stimulation sites for cognitive improvement may include the fornix, medial septal nuclei, hippocampus, entorhinal cortex, and/or temporal cortex. The therapy can be paired with events, such as but not limited to a cognitive task. The events may be manually triggered by the user (patient or caregiver) or automatically triggered by another sensor. Sensors may include wearable, external sensors and/or an internal physiological sensor. Sensors may be used to determine when to stimulate, and/or may be used to determine the stimulation and/or patterns for the stimulation. The clinical programmer may be configured with a GUI to assist with selecting the target. Examples of targets may include different DBS targets (e.g., NBM) or subregions of a DBS target, different VNS targets (e.g., cervical VNS or subregions of a VNS target), and/or different SCS targets (cervical SCS and thoracic SCS) or subregions of the SCS targets. The GUI may also include a visualization panel with a representation of therapy targets such as may be positioned with respect to an image of human anatomy.

A device used by the patient or caregiver of the patient, such as a remote control, phone or tablet, may present different tasks available for selection (e.g., different memory games or programs to learn a new skill, such as a cognitive or motor skill). The therapy or therapies provided by the system may depend on the selected tasks. However, the present subject matter is not limited to these examples.

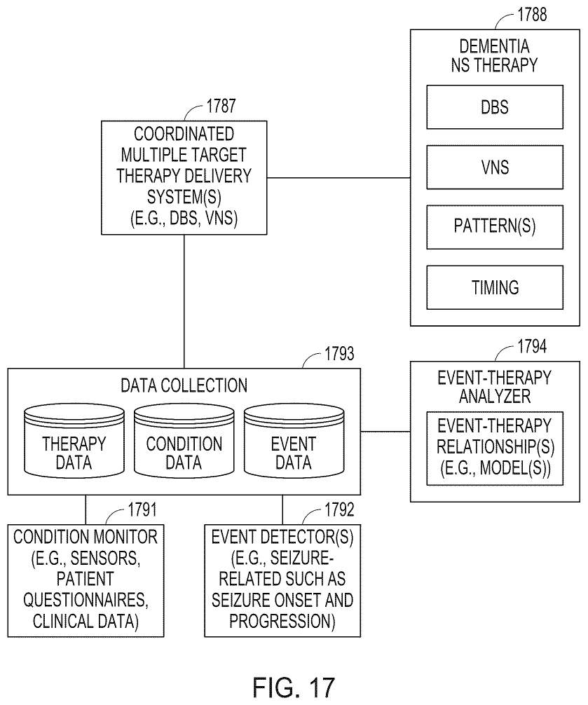

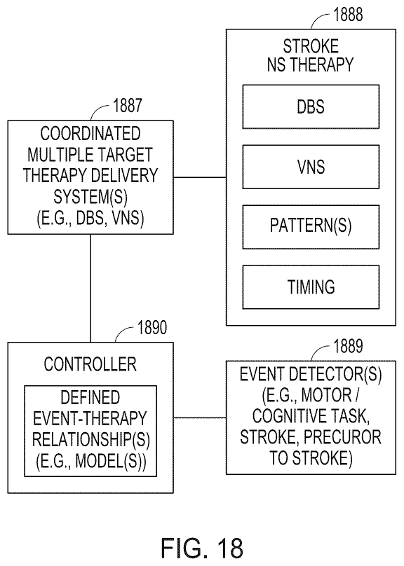

FIG. 17 illustrates, by way of example and not limitation, a system configured for defining event-therapy relationship(s) associating dementia therapies to detected dementia-related events. Similar to FIG. 16, the system includes at least one therapy delivery system 1787 configured to deliver at least two therapies 1788 to at least two therapy targets including deliver a first therapy to a first therapy target and deliver a second therapy to a second therapy target. The different therapies may include different therapy targets, different stimulation parameters (e.g., different pulse-to-pulse intervals, amplitudes, waveforms, as well as different number of pulses in different bursts of pulses, and different burst-to burst intervals, etc.), and different stimulation timing (start and stop times, as well as pulse frequency, pulse width, bursts of pulses, duty cycles between bursts, and relative timing between therapies), The therapy targets may include targets identified in FIG. 9, or may include other therapy targets. The same therapy system may be configured to deliver to the two targets (such as a DBS system configured to stimulate two or more DBS targets, a VNS system configured to stimulate two or more VNS targets, or a SCS system configured to stimulate or more SCS targets. Systems may be developed to provide one device configured to deliver stimulation to DBS target(s) and VNS target(s), to DBS target(s) and SCS target(s), to VSN target(s) and SCS target(s), or to DBS target(s), VNS target(s) and SCS target(s). Systems may be developed to provide direct communication (or indirect communication via a programmer, remote control, tablet or phone) between different devices to coordinate the therapies provided by the devices (e.g., communication links to a DBS device, to a VNS device, and/or to a SCS device. The system therapy delivery system 1787 is configured to coordinate the different therapies.

The system may include a condition monitor(s) 1691 to detect a dementia-related patient condition or the related to therapies delivered to the patient (including side effects, comorbidities, medication/medication schedule, and the like). The system may also include at least one event detector(s) 1172 to detect predefined events. Machine learning may be implemented to identify event(s) that appear to have an effect on the patient or the efficacy of the therapy (ies) delivered to the patient with dementia. The system may include a data collection system 1793 configured to detect epilepsy therapy data (e.g., therapy configuration data such as stimulation parameters, neural stimulation sites, stimulation patterns, stimulation timing, and the like) for the at least two therapy (ies), condition data from the condition monitor, and event data from the event detector(s). The event-therapy analyzer 1794 may be configured to use machine learning to analyze the collected data, event data and condition data to identify event-therapy relationship(s) (e.g., models) that may be used by the controller 1790 in the system of FIG. 16 to coordinate the dementia therapies in response to detected event(s). A plurality of events may be detected to compile event data. The event data, the therapy data, and the condition data may be analyzed to determine whether one or more of the at least two dementia therapies are effective in treating the condition when delivered in response to the one or more of the detected plurality of events, and define one or more event-therapy relationships associating the one or more of the at least two therapies to be delivered in response to the one or more of the detected events.

FIG. 18 illustrates, by way of example and not limitation, a system for treating a condition associated with stroke using multiple therapies and at least one event detector for detecting stroke-related events. The system may include at least one therapy delivery system 1887 configured to deliver at least two therapies 1888 to at least two therapy targets including deliver a first therapy to a first therapy target and deliver a second therapy to a second therapy target. The different therapies may include different therapy targets, different stimulation parameters (e.g., different pulse-to-pulse intervals, amplitudes, waveforms, as well as different number of pulses in different bursts of pulses, and different burst-to burst intervals, etc.), and different stimulation timing (start and stop times, as well as pulse frequency, pulse width, bursts of pulses, duty cycles between bursts, and relative timing between therapies). The therapy targets may include targets identified in FIG. 9 or may include other therapy targets. Targets of interest for stroke may include the cerebellum, the cortex, spinal cord, vagus nerve, internal capsule, thalamus, periventricular/periaqueductal gray, nucleus accumbens, GPi, posterior subthalamic area/zona incerta. For example, two or more therapies may be applied to a patient who had had a stroke or is determined to be at risk for having a stroke.

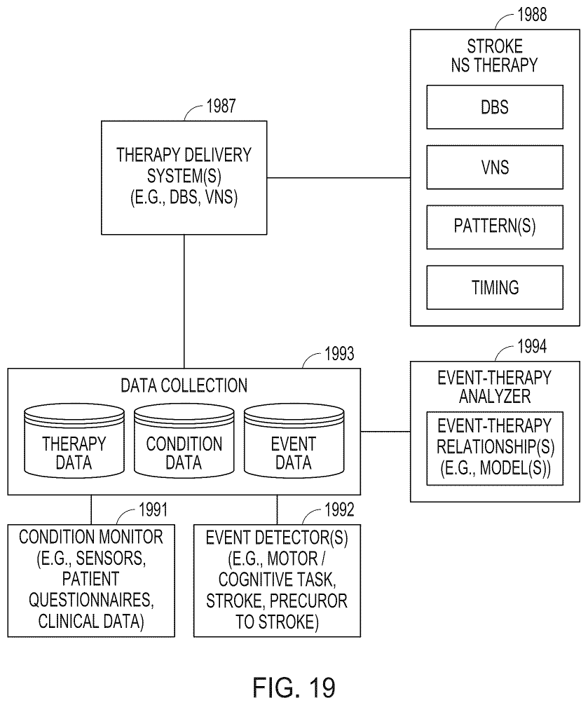

The system may include at least one event detector 1889 configured to detect at least one predefined event. The event(s) are relevant or determined to be potentially relevant to stroke. For example, events may include precursors to stroke which may be previously known or learned by the system of FIG. 19. The system may include a controller 1890 configured to coordinate the at least two therapies based on the detected at least one predefined event using at least one defined event-therapy relationship. For example, various models may be developed and used to determine the appropriate therapy (ies) that should be delivered in response to the event to treat condition(s) of the patient. The controller may be implemented in one or more of the therapy-delivery device(s) in the therapy-delivery system(s) 1887 or may be one or more separate controllers (e.g., programmer(s), remote control(s), phone(s), tablet(s) and the like) configured to communicate and with the therapy-delivery device(s) in the therapy delivery system 1887.

FIG. 19 illustrates, by way of example and not limitation, a system configured for defining event-therapy relationship(s) associating stroke therapies to detected stroke-related events. Similar to FIG. 18, the system includes at least one therapy delivery system 1987 configured to deliver at least two therapies 1988 to at least two therapy targets including deliver a first therapy to a first therapy target and deliver a second therapy to a second therapy target. The different therapies may include different therapy targets, different stimulation parameters (e.g., different pulse-to-pulse intervals, amplitudes, waveforms, as well as different number of pulses in different bursts of pulses, and different burst-to burst intervals, etc.), and different stimulation timing (start and stop times, as well as pulse frequency, pulse width, bursts of pulses, duty cycles between bursts, and relative timing between therapies), The therapy targets may include targets identified in FIG. 9, or may include other therapy targets. The same therapy system may be configured to deliver to the two targets (such as a DBS system configured to stimulate two or more DBS targets, a VNS system configured to stimulate two or more VNS targets, or a SCS system configured to stimulate or more SCS targets. Systems may be developed to provide one device configured to deliver stimulation to DBS target(s) and VNS target(s), to DBS target(s) and SCS target(s), to VSN target(s) and SCS target(s), or to DBS target(s), VNS target(s) and SCS target(s). Systems may be developed to provide direct communication (or indirect communication via a programmer, remote control, tablet or phone) between different devices to coordinate the therapies provided by the devices (e.g., communication links to a DBS device, to a VNS device, and/or to a SCS device. The system therapy delivery system 1987 is configured to coordinate the different therapies.

The system may include a condition monitor(s) 1991 to detect a patient condition that is the condition (e.g., stroke) being treated by the therapies or a condition associated with the stroke or the stroke-related therapies (including side effects, comorbidities, medication/medication schedule, and the like). The system may also include at least one event detector(s) 1992 to detect predefined events. Machine learning may be implemented to identify event(s) that appear to have an effect on the patient or the efficacy of the therapy (ies) delivered to the patient with epilepsy. The system may include a data collection system 1993 configured to detect epilepsy therapy data (e.g., therapy configuration data such as stimulation parameters, neural stimulation sites, stimulation patterns, stimulation timing, and the like) for the at least two therapy (ies), condition data from the condition monitor, and event data from the event detector(s). The event-therapy analyzer 1994 may be configured to use machine learning to analyze the collected data, event data and condition data to identify event-therapy relationship(s) (e.g., models) that may be used by the controller 1890 in the system of FIG. 18 to coordinate the epilepsy therapies in response to detected event(s). A plurality of events may be detected to compile event data. The event data, the therapy data, and the condition data may be analyzed to determine whether one or more of the at least two epilepsy therapies are effective in treating the condition when delivered in response to the one or more of the detected plurality of events, and define one or more event-therapy relationships associating the one or more of the at least two therapies to be delivered in response to the one or more of the detected events.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method for treating a condition, comprising:
using at least one event detector to detect at least one predefined event;
using a controller configured to use at least one defined event-therapy relationship and the detected at least one predefined event to coordinate at least two therapies delivered to at least two therapy targets, wherein the at least two therapies include a first therapy delivered to a first therapy target and a second therapy delivered to a second therapy target.

2. The method of claim 1, wherein the at least two therapies include a deep brain stimulation (DBS) therapy to a DBS target and one or both of a vagus nerve stimulation therapy (VNS) to a vagal nerve target or a spinal cord stimulation therapy (SCS) to a target in or near a spinal cord.

3. The method of claim 1, wherein the at least two therapies include a first deep brain stimulation (DBS) therapy to a first DBS target and a second DBS therapy to a second DBS target.

4. The method of claim 1, wherein the first therapy includes a first vagal nerve stimulation (VNS) therapy to a first VNS target and the second therapy includes a second VNS therapy to a second VNS target.

5. The method of claim 1, wherein the at least two therapies include an epilepsy therapy for an epileptic condition.

6. The method of claim 5, wherein the at least one event detector is used to detect the at least one predefined event by receiving a user input and identifying a predefined event related to the epileptic condition using the user input.

7. The method of claim 5, wherein the at least one event detector is used to detect the at least one predefined event by sensing electrical signals in a brain.

8. The method of claim 5, wherein the at least one event detector is used to detect the at least one predefined event by sensing movement or lack of movement.

9. The method of claim 5, wherein the at least one event detector is used to detect the at least one predefined event by analyzing an image of a patient to detect the predefined event related the epileptic condition.

10. The method of claim 1, wherein the at least two therapies include a deep brain stimulation (DBS) therapy to a DBS target and one or both of a vagus nerve stimulation therapy (VNS) to a vagal nerve target or a spinal cord stimulation therapy (SCS) to a target in or near a spinal cord, and the controller is used to coordinate the VNS therapy and the DBS therapy based on the detected at least one event.

11. The method of claim 10, wherein the detected at least one event includes at least a first stage and a second stage for progression of the epileptic condition, and the at least one therapy delivery system is configured to coordinate the VNS therapy and the DBS therapy to provide a first therapy for the first stage and a second therapy for the second stage.

12. The method of claim 10, wherein the detected at least one event includes a detected seizure event and a detected end to the seizure event, and the controller is used to deliver the first therapy during the seizure event and the second therapy after the seizure event.

13. The method of claim 1, wherein the at least two therapies include a dementia therapy for a dementia condition.

14. The method of claim 13, wherein the at least two therapies include a deep brain stimulation (DBS) therapy to a DBS target and a vagus nerve stimulation therapy (VNS) to a vagal nerve target, the detected at least one predefined event includes a cognitive or a motor task, and the controller is used to coordinate the DBS therapy and the VNS therapy based on the cognitive task or the motor task.

15. The method of claim 1, wherein the at least two therapies include a stroke therapy for a stroke condition.

16. A method, comprising:
delivering at least two therapies to at least two different therapy targets to provide therapy data for the at least two therapies, wherein the therapy data includes therapy configuration data;
providing condition data indicative of an effect that the delivered at least two therapies has on a treated condition;
detecting a plurality of events to compile event data;
analyzing the event data, the therapy data, and the condition data to:
determine whether one or more of the at least two therapies are effective in treating the condition when delivered in response to the one or more of the detected plurality of events; and
define one or more event-therapy relationships associating the one or more of the at least two therapies to be delivered in response to the one or more of the detected events.

17. A system for treating a condition, comprising:
at least one therapy delivery system configured to deliver at least two therapies to at least two therapy targets including deliver a first therapy to a first therapy target and deliver a second therapy to a second therapy target;
at least one event detector configured to detect at least one predefined event; and
a controller configured to coordinate the at least two therapies based on the detected at least one predefined event using at least one defined event-therapy relationship.

18. The system of claim 17, wherein the at least one therapy delivery system is configured to deliver a deep brain stimulation (DBS) therapy to a DBS target and to deliver one or both of a vagus nerve stimulation therapy (VNS) to a vagal nerve target or a spinal cord stimulation therapy (SCS) to a target in or near a spinal cord.

19. The system of claim 17, wherein the at least one therapy delivery system is configured to deliver a deep brain stimulation (DBS) therapy to at least two different DBS targets.

20. The system of claim 17, wherein the at least one therapy delivery system is configured to deliver a vagus nerve stimulation therapy (VNS) to at least two different VNS targets.
